(19) United States
(12) Patent Application Publication
(10) Pub. No.: US 2018/0371402 A1
(43) Pub. Date: Dec. 27, 2018

(54) Medium composition for producing alpha-glucosidase inhibitors fermented from Paenibacillus

(71) Applicant: Tamkang University, New Taipei City (TW)

(72) Inventors: SAN-LANG WANG, New Taipei City (TW); VAN-BON NGUYEN, Buon Ma Thuot City (VN)

(21) Appl. No.: 15/906,069

(22) Filed: Feb. 27, 2018

(30) Foreign Application Priority Data
Jun. 27, 2017 (TW) 106121366

(51) Int. Cl. C12N 1/20

(52) U.S. Cl. CPC C12N 1/20

(57) ABSTRACT

A medium composition for producing alpha-glucosidase inhibitors fermented from Paenibacillus, comprising chitin as a carbon source, proteome as a nitrogen source, and inorganic salts. Via a fermentation by Paenibacillus, it may be transferred into a medium residue having a large amount of alpha-glucosidase inhibitors. Therefore, the medium composition has the efficiency of inhibiting alpha-glucosidase.

### BACKGROUND OF THE INVENTION

### Field of the Invention

The present invention relates to a medium composition, especially to a medium for increasing the amount of alpha-glucosidase inhibitors fermented from Paenibacillus.

### Description of The Related Art

Alpha-glucosidase exists in epithelial cells in human small intestine, and facilitates the absorption of glucose in small intestine. The abnormal function of alpha-glucosidase may cause Type II diabetes, Pompe disease, and Azoospermia. Alpha-glucosidase inhibitors, α-GI, may be a reversible inhibitor to inhibit the activity of alpha-glucosidase in small intestine, to delay the process of transforming polysaccharides and disaccharides into absorbable glucose and to further slow down the elevation of blood sugar after meal.

Alpha-glucosidase inhibitor is a tier-1 hypoglycemic drug for Type II diabetes, and has a significant, fast and long lasting hypoglycemic efficiency. When a single medication cannot achieve the expected effect, it may be used along with other hypoglycemic drugs to improve high blood sugar and keep the oral administration simple.

During the research, the inventors discover that multiple bacterium from Paenibacillus sp. may produce alpha-glucosidase inhibitors more or less during the fermentation. Taiwan Application No. 105137545 discloses a Paenibacillus DSM 32521 for producing alpha-glucosidase inhibitors in an animal experiment. The alpha-glucosidase inhibitors produced from Paenibacillus DSM 32521 do not have side effects on the animals, comparing to known glucosidase inhibitors, such as Acarbose.

The present invention improves the composition of the liquid medium to enhance the growth rate of Paenibacillus to increase the production rate per unit volume, further to lower the budgets and shorten the time of manufacture.

### SUMMARY OF THE INVENTION

The purpose of the present invention is providing a medium composition for producing alpha-glucosidase inhibitors fermented from Paenibacillus. Via a fermentation by Paenibacillus, it may be transferred into a medium residue having a large amount of alpha-glucosidase inhibitors. Therefore, the medium composition has the efficiency of inhibiting alpha-glucosidase.

To achieve the aforementioned purpose, the technical feature of the present invention is providing a medium comprising chitin, inorganic salts and proteome, in which the carbon/nitrogen source of the medium is chitin and proteome individually, and the best mixture ratio of chitin and proteome is 1:0.2.

### BRIEF DESCRIPTION OF THE DRAWINGS

The detailed description of the drawings particularly refers to the accompanying figures in which:

FIG. 1 is a growth chart of Paenibacillus DSM 32521 in different medium compositions.

FIG. 2 is a bar chart of the inhibitory activity to yeast alpha-glucosidase by alpha-glucosidase inhibitors produced from different ratios of chitin and proteome.

### DETAILED DESCRIPTION OF THE INVENTION

The purpose, construction, features, functions and advantages of the present invention can be appreciated and understood more thoroughly through the following detailed description with reference to the attached drawings.

A medium composition for providing alpha-glucosidase inhibitors fermented from Paenibacillus, wherein the medium may enhance the growth of microorganism, especially when incubating Paenibacillus in the liquid medium of the present invention, it may be transformed into a medium residue having a large amount of alpha-glucosidase inhibitors via a fermentation of Paenibacillus, and the medium residue may be used to produce an oral agent for inhibiting alpha-glucosidase.

The medium of present invention comprises 0.1-2.5 wt. % carbon/nitrogen source and 0.01-0.2 wt. % inorganic salts.

Wherein the carbon/nitrogen source comprises chitin and proteome individually.

The chitin is selected from any one of an inorganic salts-free crab shell powder which is protein-free by hot alkaline process or an inorganic salts-free shrimp shell powder, wherein the chitin is 1% inorganic salts-free crab shell powder preferably.

The proteome comprises peptone and yeast extract, wherein a mixture ratio of peptone and yeast extract is from 5:3 to 7:5, and a best ratio of proteome, protein, is 6:4.

The inorganic salts of the present embodiment are MgSO4.7H2O and K2HPO4 individually, wherein a best ratio of chitin and MgSO4.7H2O is 1:0.05, and a best ratio of chitin and K2HPO4 is 1:0.1.

### Embodiment 1

Test of a mixture ratio of the carbon/nitrogen source

The present invention utilizes the mixture of 1% inorganic salts-free crab shell powder as chitin and proteome with different ratios (0/1, 0.2/1 and 0.4/1), and has the mixture fermented with Paenibacillus DSM 32521 for four days to collect alpha-glucosidase inhibitors from the supernatant.

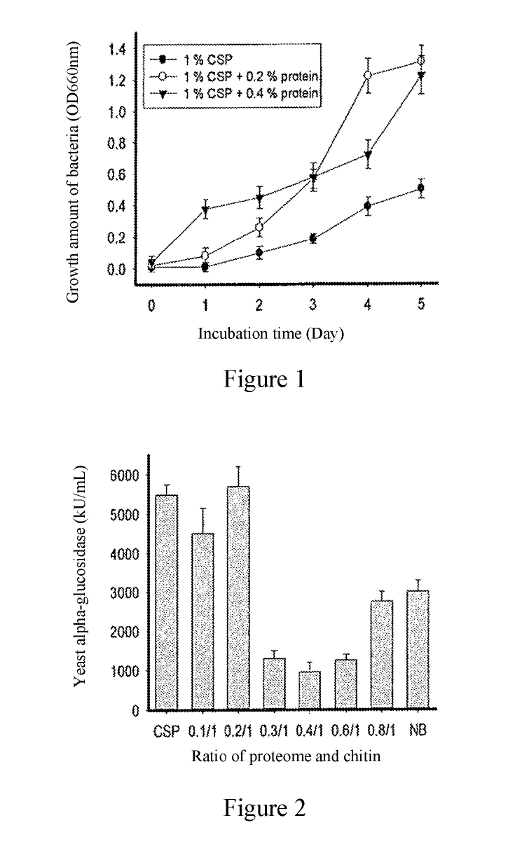

Please refer to FIG. 1, when the mixture ratio of the inorganic salts-free crab shell powder, CSP, and the proteome, protein, is 1:0.2, the activity of alpha-glucosidase inhibitors is the strongest.

Furthermore, Paenibacillus DSM 32521 is incubated in the medium having different ratios of proteome and chitin (0.1/1, 0.2/1, 0.3/1, 0.4/1, 0.6/1 and 0.8/1), and in a commercial medium NB separately. The alpha-glucosidase inhibitors produced are being tested the inhibitory activity to yeast alpha-glucosidase separately.

Please refer to FIG. 2, when the mixture ratio of proteome and chitin is 0.2:1, alpha-glucosidase inhibitors produced therein have the strongest inhibitory activity.

Alpha-glucosidase inhibitors produced by different Paenibacillus

The present experiment is utilizing the medium of the present invention to incubate different Paenibacillus and other incubations, collecting the supernatant after fermentation, and analyzing the inhibitory activities to alpha-glucosidase of yeast, bacteria, and rat respectively.

Please refer to Table 1 below, after the application of the medium of the present invention, the supernatant produced from Paenibacillus has the inhibitory activity to all alpha-glucosidases of yeast, bacteria, and rat. Therefore, the medium of the present invention may also produce alpha-glucosidase inhibitors from different Paenibacillus, but not limited to Table 1.

TABLE 1
Unit: Inhibitory rate (%)

| | | Source of | | |
|---|---|---|---|---|
| | | alpha-glucosidase | | |
| No. | Seed culture | Yeast | Bacteria | Rat |
| 1 | Bacillus licheniformis TKU004 | — | — | — |
| 2 | Bacillus subtilis TKU007 | — | — | — |
| 3 | Bacillus mycodides TKU038 | 12 | — | 8 |
| 4 | Bacillus mycodildes TKU040 | — | — | 9 |
| 5 | Paenibacillus macerans TKU029 | 96 | 90 | 92 |
| 6 | Paenibacillus mucilaginosus TKU032 | 98 | 95 | 95 |
| 7 | Paenibacillus sp. TKU037 | 99 | 95 | 90 |
| 8 | Paenibacillus sp. TKU042 | 99 | 97 | 96 |
| 9 | Serratia marcescens TKU011 | — | — | — |
| 10 | Serratia ureilytica TKU013 | — | — | — |
| 11 | Serratia | — | — | — |
| | marcescens subsp. sakuensis TKU019 | | | |
| 12 | Lactobacillus | — | — | 7 |
| | paracasei subsp. paracasei TKU010 | | | |
| 13 | Pseudomonas tamsuii TKU015 | — | — | — |
| 14 | Serratia sp.TKU016 | — | — | — |
| 15 | Serratia sp.TKU020 | — | — | — |
| 16 | Bacillus Cereus TKU033 | — | — | 15 |
| 17 | Rhizobium sp. TKU041 | — | — | — |

| | | | |
|---|---|---|---|
| Control group without bacteria | — | — | — |

*The name of bacteria followed by TKUXXX is the seed culture screened by the inventors, however, not the claimed seed culture.

Embodiment 2

The present embodiment focuses on the comparison of the amount of alpha-glucosidase inhibitors produced from Paenibacillus in the present medium and the commercial medium NB separately. In the present embodiment, Paenibacillus DSM 32521 is incubated in the present medium and the commercial medium NB individually, and the alpha-glucosidase inhibitors produced therein are being tested for their inhibitory activity to alpha-glucosidase of S. cerevisiae.

Please refer to Table 2 below, in the comparison between the present medium and the commercial medium NB, the production rate of alpha-glucosidase inhibitors increases by 2.5 times, from 5,000 U/mL to12,379 U/mL, and the value of IC50 reduces by 12 times, from 81 μg/mL to 6.7 μg/mL. It indicates that the present medium increases the growth rate of microorganism, the bacteria amount per unit volume, and the production rate of alpha-glucosidase inhibitors produced from Paenibacillus fermentation.

TABLE 2

| | Commercial medium | Medium of the present |
|---|---|---|
| Comparative factors | NB | invention |
| Carbon/nitrogen | Nutrient broth | Inorganic salts-free crab |
| source | | shell powder, CSP |
| Incubation temperature | 30 | 30 |
| (° C.) | | |
| Carbon/nitrogen | 0.8 | 1.6 |
| concentration (%) | | |
| Incubation time | 4 (Day) | 4 (Day) |
| Ratio of medium and | 100/250 | 130/250 |
| culture flask | | |
| Seed culture (%) | 1 | 1-4 |
| IC₅₀ (μg/mL) | 81 ± 4.3 | 6.7 ± 0.31 |
| Production rate of | 5000 | 12379 |
| alpha-glucosidase | | |

Embodiment 3

The present embodiment is a comparison between alpha-glucosidase inhibitors fermented from Paenibacillus DSM 32521 in the present medium and the commercial diabetes drug, Acarbose, specific to the inhibitory activity to various alpha-glucosidases.

Please refer to Table 3 below, the alpha-glucosidase inhibitors produced by the present medium have a stronger inhibitory activity to rat alpha-glucosidase than the commercial diabetes drug, Acarbose3

TABLE 3

| | | DSM 32521 | | Acarbose | |
|---|---|---|---|---|---|
| | | | Maximum | | Maximum |
| Source of | | IC₅₀ | Inhibitory | IC₅₀ | Inhibitory |
| alpha-glucosidase | n | (μg/mL) | Activity (%) | (μg/mL) | Activity (%) |
| S. cerevisiae | 3 | 6.7 ± 0.31 | 99 ± 2.2 | 1095 | 74 ± 3.4 |
| α-glucosidase | | | | | |
| Rat α-glucosidase | 3 | 15.9 ± 0.7 | 97 ± 2.7 | 78 ± 3.2 | 91 ± 3.1 |
| B. stearothermophilus | 3 | 6.6 ± 0.22 | 95 ± 2.3 | 0.042 ± 0.003 | 99 ± 1.7 |
| α-glucosidase | | | | | |
| Rice α-glucosidase | 3 | 6.7 ± 0.25 | 96 ± 1.9 | 3.04 ± 0.82 | 100 ± 2.1 |

### SYMBOL DESCRIPTION

CSP inorganic salts-free crab shell powder

NB commercial medium

### Deposit of Biological Material

The original biomaterial of Paenibacillus DSM 32521 is deposited as Issue Number U.S. Pat. No. 9,850,519.

What is claimed is:

1. A medium composition for producing alpha-glucosidase inhibitors fermented from Paenibacillus, comprising 0.1-2.5 wt. % carbon/nitrogen source and 0.01-0.2 wt. % inorganic salts, wherein the medium composition provides alpha-glucosidase inhibitors fermented from Paenibacillus.

2. The medium composition of claim 1, wherein the carbon source and the nitrogen source are a chitin and a proteome individually.

3. The medium composition of claim 2, wherein the chitin is selected from any one of an inorganic salts-free crab shell powder which is protein-free and an inorganic salts-free shrimp shell powder which is protein-free.

4. The medium composition of claim 2, wherein the proteome comprises a peptone and a yeast extract.

5. The medium composition of claim 1, wherein the inorganic salts comprise MgSO₄.7H₂O and K₂HPO₄.

6. The medium composition of claim 2, wherein a best mixture ratio of the chitin and the proteome is 1:0.2.

7. The medium composition of claim 4, wherein a mixture ratio of the peptone and the yeast extract is from 5:3 to 7:5, and a best mixture ratio of the peptone and the yeast extract is 6:4.

8. The medium composition of claim 2, wherein a best mixture ratio of the chitin and MgSO₄.7H₂O is 1:0.05.

9. The medium composition of claim 5, wherein a best mixture ratio of the chitin and MgSO₄.7H₂O is 1:0.05.

10. The medium composition of claim 2, wherein a best mixture ratio of the chitin and K₂HPO₄ is 1:0.1.

11. The medium composition of claim 5, wherein a best mixture ratio of the chitin and K₂HPO₄ is 1:0.1.
